(19) United States
(12) Patent Application Publication
(10) Pub. No.: US 2005/0283123 A1
(43) Pub. Date: Dec. 22, 2005

(54) AVAP: air valve and port for intravenous (IV) tubing

(76) Inventors: Felicia C. Lyde, Brooklyn, NY (US); Cassandra Ann Gills, Virginia Beach, VA (US)

Correspondence Address: Attention: PATENTS;COWAN, LIEBOWITZ & LATMAN, P.C., 1133 AVENUE OF THE AMERICAS, NEW YORK, NY, 10036, US

(21) Appl. No.: 11/155,250

(22) Filed: Jun. 17, 2005

Related U.S. Application Data
(60) Provisional application No. 60/580,870, filed on Jun. 18, 2004.

(51) Int. Cl. A61M 5/00

(52) U.S. Cl. 604/254

(57) ABSTRACT

The invention provides an air valve for use in getting air out of intravenous (IV) tubing and preventing the air from intravenous (IV) tubing from entering healthcare patients' veins. In an illustrative embodiment the air valve is attached at one end to the intravenous (IV) tubing that hangs from an intravenous (IV) bag and at the other end to the injection site, where a needle is inserted into a patient's vein. The invention prevents any air from entering a healthcare patient's blood stream and causing air embolism, a potentially lethal complication caused by an accumulation of air bubbles in the blood stream that hinders the circulation of blood. The invention will also lead to increased productivity of intravenous (IV) drips since it allows for a seamless interchange of medications and solutions.

### BACKGROUND OF THE INVENTION

There is no prior technology for getting air out of intravenous (IV) tubing and preventing the air from intravenous (IV) tubing from entering healthcare patients' veins. Presently, healthcare practitioners tap intravenous (IV) tubing with their fingernails to prime new intravenous (IV) tubing and to push air out of the intravenous (IV) tubing. If healthcare practitioners cannot get the air out of the intravenous (IV) tubing, then they must prime new intravenous (IV) tubing; however, this leads to unnecessary multiple injections to healthcare patients' veins.

According to Dr. John Peter Gruen of the Department of Neurosurgery at the University of Southern California, air embolism is “the entry of air into the blood stream with formation of bubbles that can reach sufficient volume that they interfere with normal pumping of blood by the heart.” Moreover, according to Dr. T. Kapoor and Dr. G. Gutierrez of the Department of Internal Medicine at George Washington University, air embolism is “a cause of the systemic inflammatory response syndrome.” Although many occurrences of air embolism are asymptomatic, entrainment of large quantities of air can lead to cardiovascular collapse, severe neurological injury, and even death. The mechanism of death from massive air embolism is circulatory obstruction and cardiovascular collapse resulting from air trapped in the right ventricular outflow tract.

Venous air embolism that does not cause immediate death can cause paradoxical embolization by acutely increasing right atrial pressure resulting in right to left shunt through a patent foramen ovale. Pulmonary microvascular occlusion can also occur; the air can produce increasing obstruction to blood flow, undergo resorption, or result in increased dead space. Bronchoconstriction may result from release of endothelial mediators, complement production, and cytokine release. Bronchoconstriction results in increased airway pressure and wheezing. During spontaneous respiration, slow entrainment of air that causes obstruction of 10% of the pulmonary circulation causes a “gasp” reflex that results in chest pain and tachypnea. The resulting decrease in intrathoracic pressure and right atrial pressure can increase the rate of air entrainment. Other manifestations of air embolism include hypoxemia, hypercapnia, and decreased ETCO2 (end-tidal carbon dioxide) due to increased functional dead space. Hypotension, cardiac dysrhythmias, and cardiovascular collapse occur as air entrainment continues.

Morbidity and mortality from air embolism are directly related to the size of the embolus and the rate of entry. Doses of air greater than fifty milliliters (50 ml) or one milliliter per kilogram (1 ml/kg) cause hypotension and dysrhythmias. Three hundred milliliters (300 ml) of air entrained rapidly can be lethal.

U.S. Pat. No. 6,261,267 of Chen discloses an IV container spike and drip chamber assembly, which includes an automatic shut off mechanism to allow nurses to change the fluid container without removing the catheter from the patient. The automatic shut off mechanism comprises a floating ball capable of being supported by or floating in the fluid due to the buoyancy of the fluid. The IV container spike also includes an air vent which allows the air in the drip chamber to vent when nurses want to change the container of medical fluid. As the medicinal fluid is gradually reduced in level in the drip chamber, the floating ball is lowered thereby and when the floating ball reaches the bottom of the drip chamber, the floating ball blocks the outlet so as to stop the injection.

### OBJECTS OF THE INVENTION

It is an object of the invention to provide a mechanism to take air out of intravenous (IV) tubing and keep air out of healthcare patients' veins, i.e., air that has built up either from: 1) the solution or medication running low from the intravenous (IV) bag, or 2) improper priming of the initial intravenous (IV) drip.

It is also an object of this invention to allow healthcare practitioners to change intravenous (IV) bags without removing the needle from the patient's vein in order to prime new intravenous (IV) tubing.

### SUMMARY OF THE INVENTION

This invention solves a healthcare safety issue, i.e., air embolism is a potentially lethal complication caused by preventable negligence. Air embolism is when air bubbles accumulate in the blood stream and hinder the circulation of blood. This invention prevents any air from entering a healthcare patient's blood stream and causing air embolism, also known as venous air embolism.

Use of this invention will lead to increased productivity of intravenous (IV) drips because the invention allows for a seamless interchange of medications and solutions. Moreover, use of the invention will decrease the unproductive employment of healthcare practitioners' skills and decrease the overburdening of healthcare practitioners' time. Therefore, use of this invention will lead to a more effective use of healthcare practitioners' time and talents and to the effective prevention of air embolism.

In accordance with these and other aspects of the invention, there is disclosed a valve for controlling the flow of fluid into a patient and preventing the introduction of air into the patient. The valve comprises a chamber comprising a bottom and a top base of the chamber. The top base is attached to the top of the chamber, has a top opening there through, and is adapted to facilitate the flow of the fluid through the top opening and the chamber. Further, a bottom base is attached to the bottom of the chamber and has a bottom opening disposed there through. A sphere is disposed within the chamber, whereby the sphere is adapted to float upon the fluid introduced, and is dimensioned and configured with respect to the bottom opening to prevent the discharge of air from the chamber and through the bottom opening when there is no fluid in the chamber and the sphere is disposed in its blocking position with respect to the bottom opening. Further, a stop mechanism is provided for preventing the sphere from blocking the top opening when the chamber is filled with the fluid. A mechanism is disposed adjacent the bottom opening to facilitate the withdrawal of air flowing through the chamber to enhance the amount of air that may be withdrawn from the chamber.

In a further aspect of this invention, a fluid valve is disclosed for preventing the flow of air into a patient. The valve comprises a chamber, which includes a top and a bottom. The top has a top opening introducing the fluid into the chamber and the bottom has a bottom opening for discharging the fluid from the chamber and into the patient. Further, a sphere is disposed in the chamber and adapted to rise and fall therein as the fluid is respectively introduced into or discharged from chamber, whereby the bottom opening is locked to prevent the discharge of air from the chamber and into the patient. A mechanism is provided to prevent the sphere from blocking the top opening and capturing air in the chamber when the chamber is filled with the fluid. The mechanism is disposed in a relatively close position to the upper door to enhance the free flow of fluid through the chamber.

In a still further aspect of this invention, the valve includes the bottom base, which is adapted to be directly connected to a catheter.

### BRIEF DESCRIPTION OF THE DRAWINGS

For a better understanding of the invention and further features thereof, reference is made to the following detailed description of the invention to be read in conjunction with the accompanying drawings, wherein:

FIG. 1 is a detailed view of an illustrative embodiment of the invention;

FIG. 2 is a cross-sectional view of an illustrative embodiment of the invention; and

FIG. 3 is an assembly view of an illustrative embodiment of the invention, wherein the connection of this invention to its related apparatus is illustrated.

### DETAILED DESCRIPTION OF A PREFERRED EMBODIMENT OF THIS INVENTION

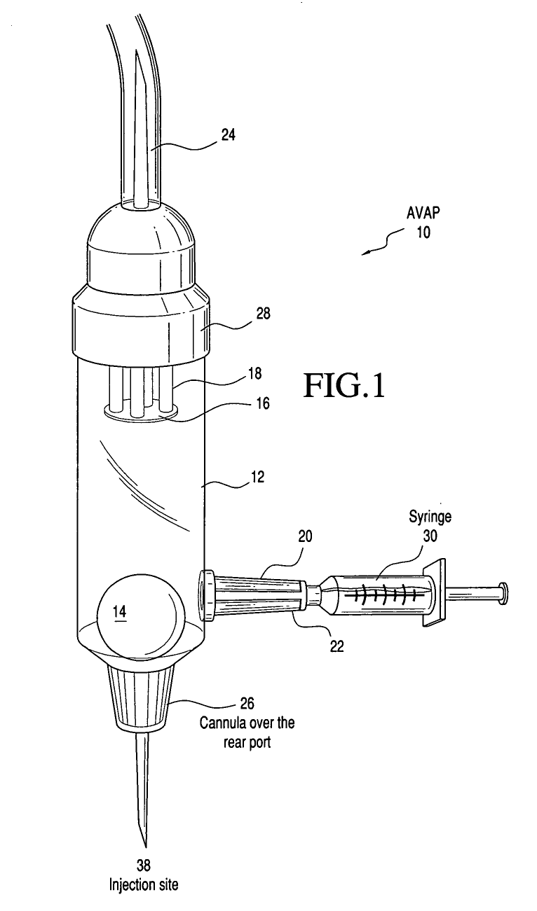

Referring now to the drawings and in particular to FIG. 1, there is shown an illustrative embodiment of an air valve 10, which may be used to extract air from intravenous (IV) tubing and prevent any air from being introduced into a patient's veins. Air valve 10 comprises a universal cylindrical drip chamber 12 with a free-floating sphere 14 inside, a horizontal circular ring 16 attached to four posts 18 that extend from the top of the inside of the drip chamber, and an external port 20 with a removable cap-cover 22 at the base of valve 10. As will be explained, the ring 16 and posts 18 form a stop mechanism to prevent the sphere 14 from blocking. The air valve 10 also comprises a spike 24 at the top of the cylindrical drip chamber 12, having a top base 28, and a bottom base 26 at the bottom of the cylindrical drip chamber 12.

In an illustrative embodiment, all parts of the valve may be made by any of the following plastic polymers: polyethylene; (non-DEHP) PVC; or (non-Di-2-Ethylhexl Phthalate) Polyvinyl Chloride.

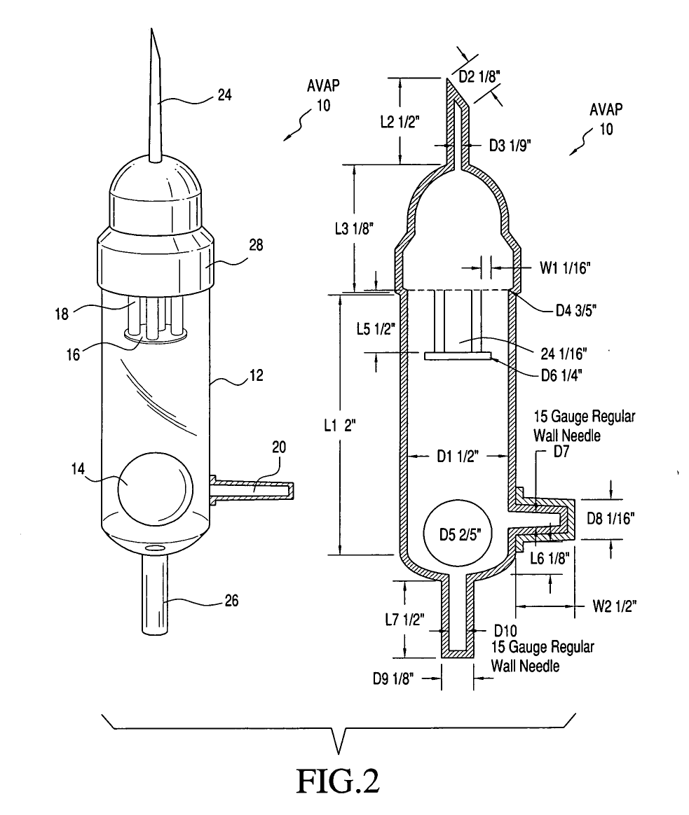

Referring now to FIG. 2, there is shown a cross-sectioned view of the illustrative embodiment with illustrative dimensions of the air valve 10 as shown in FIG. 1. Even though illustrative dimensions are set out in FIG. 2, these dimensions are not shown to scale. The cylindrical drip chamber 12 measures one-half (½″) inch in internal diameter D1 and two (2″) inches in length L1. The hollowed spike 24 at the top of the cylindrical drip chamber 12 measures one-eighth (⅛″) inch in external diameter D2 and one-half (½″) inch in length L2 and has an internal diameter of one-ninth ( 1/9″) inch D3. The spike 24 has a base 28 measuring three-fifth (⅗″) inch in diameter D4 and one-eighth (⅛″) inch in height L3 at the top of the cylindrical drip chamber 12. The sphere 14, measuring two-fifth (⅖″) inch in diameter (D5), is free-floating inside the cylindrical drip chamber 12 and is hollow in order to ensure a net upward buoyant force regardless of the type of liquid that may be in the drip chamber. The horizontal circular ring 16 measures one-fourth (¼″) inch in diameter D6 and one-sixteenth ( 1/16″) inch in thickness L4. It is suspended one-half (½″) inch L5 down from the spike base 28. The four posts 18, which measure one-sixteenth ( 1/16″) inch in width W1 and one-half (½″) inch in length L5, are connected to the inside of the spike base 28 at their top ends and attached to the horizontal circular ring 16 at their bottom ends. The external port 20, measuring one-half (½″) inch in width W2 and having an internal diameter equal to an 15 gauge regular wall needle D7 and an external diameter of one-sixteenth ( 1/16″) inch D8, is located on the side of the cylindrical drip chamber 12, one-eighth (⅛″) inch L6 from the bottom of the cylindrical drip chamber 12. The external port 20 comprises a removable cap-cover 22 having an internal diameter of one-sixteenth ( 1/16″) inch D8. The rear port 26 on the underside-bottom of the cylindrical drip chamber 12 measures one-half (½″) inch in length L7 and one-eighth (⅛″) inch in external diameter D9, and has an internal diameter equal to an 15 gauge regular wall needle D10.

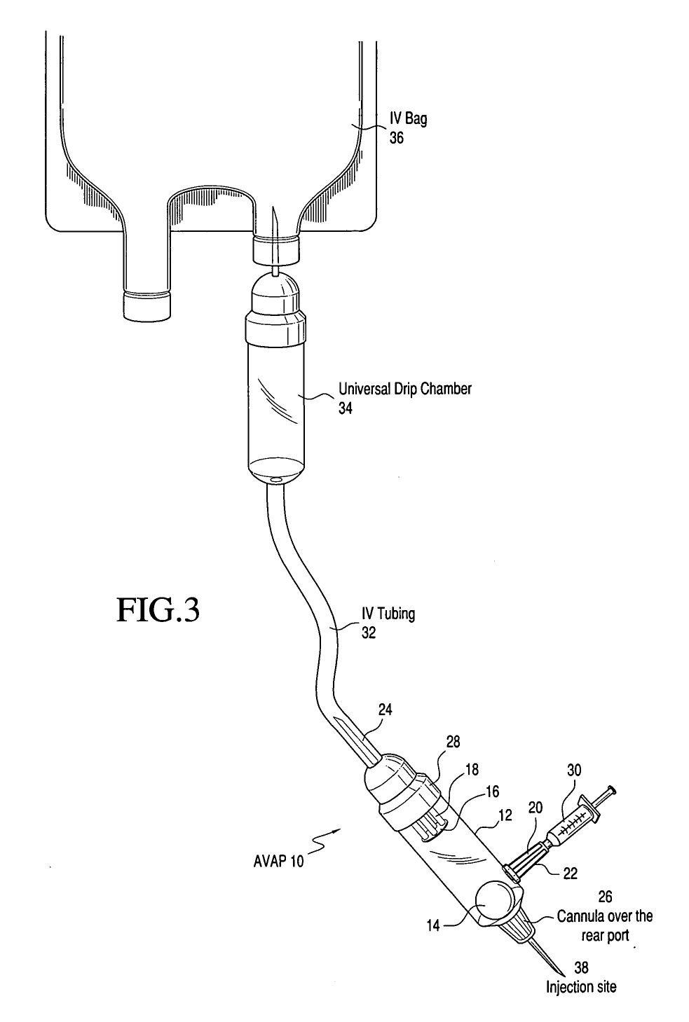

Referring now to FIG. 3, there is shown an illustrative embodiment of a connection of the air valve 10 to an intravenous (IV) bag 36. The air valve 10 is attached at one end to the intravenous (IV) tubing 32 so that the air valve 10 is directly above the injection site 38, where a needle is inserted into a patient's vein, or above the cannula that is attached to a needle that is inserted into a patient's vein. The spike 24 of the air valve 10 is inserted directly into the lowest end of the intravenous (IV) tubing 32 that hangs from the intravenous (IV) bag 36. The rear port 26 of air valve 10 is inserted directly into the injection site 38 or into a Male Luer Lock Adapter attached to a Lever Lock Cannula that is connected to the injection site 38. The cap-cover 22 for the external port 20 is removed when the external port 20 is being accessed and replaced over the external port 20 when the external port 20 is not being accessed. The Wen U.S. Pat. No. 6,261,267 of Wen discloses in contrast the direct connection of the needle to the bottom base or cannula 26, that his needle interconnected by tubing, a roller clamp and a luer connection to the Wen's needle or catheter. The advantage of direct connection of the needle to the bottom baser of this invention, as compared to Wen's device, is that more air can be trapped in the extended fluid conduit as constructed by Wen.

The sphere 14 inside the air valve 10 floats when there is sufficient liquid inside the drip chamber 12 to lift the sphere from its seat. Since the sphere 14 is hollow, a net upward buoyant force is formed when the drip chamber 12 is filled with liquid, making the sphere 14 float and thereby allowing solution or medication to pass through an opening in the bottom base 26 to the patient. The sphere 14 seals off the drip chamber 12 from the bottom base 26 when there is no liquid inside the drip chamber 12. Since the sphere 14 is denser than air but less dense than the solution or medication, the weight of the sphere 14 makes it fall when there is no liquid inside the drip chamber 12. And since the sphere 14 is greater in diameter than the rear port 26 that leads to the injection site 38, the sphere 14, when it falls, prevents any air from passing through the bottom base 26 to the patient. The horizontal circular ring 16, which is attached to the four posts 18, acts as a float stop for the sphere 14. When the sphere 14 is floating, the horizontal circular ring 16 stops the sphere 14 from floating all the way to the top of the drip chamber 12 and thereby does not allow the sphere 14 to seal off the solution or medication flowing through the spike 24 at the top of the drip chamber 12. Since the “float-stop” is composed of posts instead of a cylinder or casing, liquid can flow from the spike 24 pass around the four posts 18 and continue around the sides of the ring 16 into the drip chamber 12, regardless of the position of the sphere 14.

The U.S. Pat. No. 6,261,267 of Wen discloses, as noted above, an automatic shut off valve, and comprises a ball and a flange which keeps the ball from floating to the top when the fluid is introduced into the drip chamber. In the Wen patent, the flange in disposed adjacent the lower opening through which the fluid is discharged from the drip chamber. On the other hand, there is shown in FIGS. 2 and 3 of this document the stop mechanism comprising the posts 18 and the ring 16 which is disposed at least closer to the top opening of the top base 28, than to the bottom based 26. The flange of the Wen patent may, if the valve is disposed in another than vertical position, may lodged to block or to interrupt the fluid flow. In contrast, the stop mechanism of this invention is disposed more remotely from the bottom base 26 than the top base 28 and will not interrupt the fluid through the chamber and the opening in the bottom base 26.

The external port 20 extracts air from the intravenous (IV) tubing 32 with the use of a syringe 30. When solution or medication begins to enter the intravenous (IV) tubing 32 from the intravenous (IV) bag 36, a healthcare practitioner will insert a syringe 30 with a regular wall needle (illustratively 15 gauge) into the external port 20 and extract the air from the air valve 10 until there is only solution or medication remaining in the intravenous (IV) tubing 32 and the air valve 10. The cap-cover 22 for the external port 20 will be removed when the external port 20 is being accessed in order to insert a syringe with a regular wall needle to extract the air from the air valve 10. The cap cover 22 is then replaced over the external port 20 when the external port 20 is not being accessed, thereby preventing any air from entering the air valve 10 through the external port 20. The spike 24 makes a seamless interchange between the air valve 10 and the intravenous (IV) tubing 32 attached to the hanging intravenous (IV) bag 36; it acts as a conduit for the solution or medication flowing from the hanging intravenous (IV) bag 36 and attached intravenous (IV) tubing 32 into the air valve 10. The drip chamber 12 contains the sphere 14 that prevents any air from passing into the patient and the horizontal circular ring 16 attached to the four posts 18 that prevents the sphere 14 from floating to the top and sealing off the solution or medication flowing from the hanging intravenous (IV) bag 36 and attached intravenous (IV) tubing 32. The drip chamber 12 also collects the solution or medication which flows through the spike 24 from the hanging intravenous (IV) bag 36 and attached intravenous (IV) tubing 32 and allows the sphere 14 to float. The rear port 26 makes a seamless interchange between the air valve 10 and the injection site 38; it acts as a conduit for the solution or medication flowing from the air valve 10 into the injection site 38 and then into the patient's vein.

The U.S. Pat. No. 3,261,267 of Wen also discloses a vent that permits the release of air. This vent is disposed on the top of Wen's value and remotely from its automatic shut off mechanism. In contrast, the invention disclosed by this document positions its air release mechanism in the form of the external port 20 as shown in FIGS. 2 and 3 as being disposed adjacent the bottom base 23. The advantage of this placement is that the medical personnel operating the syringe can extract air more efficiently as the fluid is being introduced into its drip chamber 12. As the fluid is being pored into the chamber, it initially traps the air at the bottom of the chamber 12, whereby it is relatively available to be with drawn by a syringe that is inserted into the port 20, i.e., at the bottom of the chamber 12. Only after the fluid displaces the air from the bottom of the chamber 12 and rises to the top of the chamber, can the vent of the Wen device begin to withdraw the air from its chamber thereby slowing its process for air removal.

Although the present invention has been described in terms of various embodiments, it is not intended that the invention be limited to these embodiments. Modification within the spirit of the inventions will be apparent to those skilled in the art. The scope of the present invention is defined by the claims that follow.

1. A valve for controlling the flow of fluid into a patient and preventing the introduction of air into the patient, said valve comprising:
a) a chamber comprising a bottom and a top of said chamber;
b) a top base being attached to said top of said chamber, having a top opening there through, and being adapted to facilitate the flow of the fluid through said top opening and said chamber;
c) a bottom base attached to said bottom of said chamber and having a bottom opening disposed there through;
d) a sphere disposed within said chamber, said sphere being adapted to float upon the fluid introduced into said chamber, and being dimensioned and configured with respect to said bottom opening to prevent the discharge of air from said chamber and through the bottom opening when there is no fluid in said chamber and said sphere is disposed in its blocking position with respect to said bottom opening;
e) a stop mechanism for preventing said sphere from blocking said top opening when said chamber is filled with the fluid; and
f) a mechanism disposed adjacent said bottom opening to facilitate the withdrawal of air flowing through said chamber to enhance the amount of air that may be withdrawn from said chamber.

2. The valve as claimed in claim 1, wherein said stop mechanism for preventing the introduction of air into the patient comprises a stop, and at least one support for disposing said stop to prevent said sphere from blocking said top opening.

3. The valve as claimed in claim 2, wherein said stop mechanism comprises a ring and at least one member attached at one end to said top base and, at the other end to said ring, said ring and said member being adapted such that said ring is spaced sufficiently from said top base to insure the free flow of the liquid there through and also to prevent said sphere from being disposed to block said top opening.

4. The valve as claimed in claim 1, wherein there is further included a port, which is disposed relatively close to said bottom base, whereby after the fluid has been introduced into said chamber, any air still present in the fluid may be removed by accessing said port with a syringe needle.

5. The valve as claimed in claim 4, wherein said valve further includes a cap cover to be disposed about said port to further prevent the introduction of air through said port and into said chamber.

6. The valve as claimed in claim 5, wherein said valve further comprises a top spike, which is attached to said top base to permit the introduction of fluid from a container through a tubing and said needle into said chamber.

7. The valve as claimed in claim 6, wherein said valve comprises a bottom spike which is supported by said port and is in communication with said bottom opening and the patient.

8. A fluid value for preventing the flow of air into a patient, said valve comprising:
a) a chamber comprising a top and a bottom, said top having a top opening introducing the fluid into said chamber and said bottom having a bottom opening for discharging the fluid from said chamber and into the patient;
b) a sphere disposed in said chamber and adapted to rise and fall therein as the fluid is respectively introduced into or discharged from chamber, and to block said bottom opening to prevent the discharge of air from said chamber and into the patient; and
c) a mechanism to prevent said sphere from blocking said top opening and capturing air in said chamber when said chamber is filled with the fluid, said mechanism being disposed in a relative close position to said upper door to enhance the free flow of fluid through said chamber.

9. The valve as claimed in claim 8, wherein said bottom base is adapter to be directly connected to a cannula.
